(19) United States
(12) Patent Application Publication
(10) Pub. No.: US 2002/0072789 A1
(43) Pub. Date: Jun. 13, 2002

(54) Soc lubricant filler port

(76) Inventors: Steven S. Hackett, Maple Grove, MN (US); Fernando DiCaprio, Mendota Heights, MN (US); Kyle P. Taylor, Brooklyn Park, MN (US); Jan D. Seppala, Maple Grove, MN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 09/735,325

(22) Filed: Dec. 12, 2000

(51) Int. Cl. A61F 2/06

(52) U.S. Cl. 623/1.12

(57) ABSTRACT

A stent delivery system having one or more stent retaining sleeves. The at least one sleeve having at least one lubricant application port, the at least one lubricant application port defining an opening through the at least one sleeve. The opening constructed and arranged to allow a lubricious substance to pass through the at least one sleeve and at least partially coat a portion of the balloon and/or stent positioned thereunder.

### BACKGROUND OF THE INVENTION

1. Field Of The Invention

This invention relates to a medical device delivery systems, namely catheter mounted stent delivery systems. More particularly, the present invention is directed to socks or sleeves used to retain a stent on a stent delivery catheter. The present invention provides for one or more stent end retaining sleeves having one or more lubricant filler ports which may be used to apply lubricant to the balloon cone and or waist without excess wicking of lubricant onto the balloon body and stent.

2. Description Of The Related Art

Stents and stent delivery assemblies are utilized in a number of medical procedures and situations, and as such their structure and function are well known. A stent is a generally cylindrical prosthesis introduced via a catheter into a lumen of a body vessel in a configuration having a generally reduced diameter and then expanded to the diameter of the vessel. In its expanded configuration, the stent supports and reinforces the vessel walls while maintaining the vessel in an open, unobstructed condition.

Both self-expanding and inflation expandable stents are well known and widely available in a variety of designs and configurations. Self-expanding stents must be maintained under a contained sheath or sleeve(s) in order to maintain their reduced diameter configuration during delivery of the stent to its deployment site. Inflation expandable stents are crimped to their reduced diameter about the delivery catheter, then maneuvered to the deployment site and expanded to the vessel diameter by fluid inflation of a balloon positioned between the stent and the delivery catheter. The present invention is particularly concerned with delivery and deployment of inflation expandable stents, although it is generally applicable to self-expanding stents when used with one or more stent retaining sheaths.

In advancing an inflation expandable stent through a body vessel to the deployment site, there are a number of important considerations. The stent must be able to securely maintain its axial position on the delivery catheter without translocating proximally or distally and especially without becoming separated from the catheter. The stent, particularly its distal and proximal ends, must be protected to prevent distortion of the stent and to prevent abrasion and/or reduce trauma of the vessel walls.

Inflation expandable stent delivery and deployment assemblies are known which utilize restraining means that overlie the stent during delivery. U.S. Pat. No. 4,950,227 to Savin et al., relates to an inflation expandable stent delivery system in which a sleeve overlaps the distal or proximal margin (or both) of the stent during delivery. During inflation of the stent at the deployment site, the stent margins are freed of the protective sleeve(s). U.S. Pat. No. 5,403,341 to Solar, relates to a stent delivery and deployment assembly which uses retaining sheaths positioned about opposite ends of the compressed stent. The retaining sheaths of Solar are adapted to tear under pressure as the stent is radially expanded, thus releasing the stent from engagement with the sheaths. U.S. Pat. No. 5,108,416 to Ryan et al., describes a stent introducer system which uses one or two flexible end caps and an annular socket surrounding the balloon to position the stent during introduction to the deployment site.

A common problem which occurs in catheter assemblies is friction or adhesion between various parts which periodically come into contact with one another during the medical procedure. For instance, friction can occur between the guide catheter and guide wire, between the introducer sheath and the guide catheter, or between the guide catheter and the balloon catheter, for instance, and may increase the difficulty of insertion, cause loss of catheter placement, and result in discomfort to the patient or damage to the vasculature. In catheters equipped with stent retaining socks or sleeves, friction between the balloon and sleeve, and/or the stent and sleeve may also cause retraction of the sleeves to be made more difficult. It is therefore desirable to reduce the friction due to the sliding between the various parts of the catheter assemblies. Copending U.S. application Ser. No. 09/549,286 which was filed Apr. 14, 2000 describes a reduced columnar strength stent retaining sleeve having a plurality of holes. The relatively reduced columnar and radial strength provided by the holes allows the sleeve to be retracted off of a stent without the need for lubricant.

The materials from which catheters are produced are typically polymeric or metallic in nature, and in general, are inherently non-lubricious. When these non-lubricious materials come into contact, friction occurs. Medical device manufacturers have used various approaches to reduce the coefficient of friction between these surfaces.

Lubricants of many types have been used in conjunction with balloon catheters. Both hydrophilic and hydrophobic coatings and lubricants are well known in the catheter art. The present invention may be used in conjunction with any type of lubricious substance suitable for use with a stent delivery catheter, and is further directed to the application of the lubricious substance to the surface of a balloon cone and/or waist subsequent to stent mounting and sleeve placement onto the catheter.

Copending U.S. patent application Ser. No. 09/407,836 which was filed on Sep. 28, 1999 and entitled Stent Securement Sleeves and Optional Coatings and Methods of Use, provides for a stent delivery system having sleeves. In 09/407,836 the sleeves may be made up of a combination of polytetrafluoroethylene (hereinafter PTFE) as well as one or more thermoplastic elastomers. Copending U.S. patent application Ser. No. 09/427,805 filed Oct. 27, 1999, and entitled End Sleeve Coating for Stent Delivery, describes the use of stent retaining sleeves having lubricious coatings applied thereto. Copending U.S. patent application Ser. No. 09/273,520 filed Mar. 22, 1999, entitled Lubricated Sleeve Material For Stent Delivery likewise describes the use of stent retaining sleeves and lubricants.

Unlike the various references cited herein, the present invention is directed to a method of applying any type of lubricious substance to the surface of a balloon cone, even after the stent and stent retaining sleeves are mounted to the catheter.

### BRIEF SUMMARY OF THE INVENTION

The present invention is directed to providing a stent retaining sleeve with one or more lubricant application ports. The lubricant port(s) allow lubricant application to the balloon cone after the sleeve is mounted on to the stent delivery catheter. The port(s) may be positioned along the portion of the waist and/or the cone portion of the sleeve. Where the port(s) are located on the cone portion, or extend to the cone portion, the port(s) may be constructed to provide controlled sleeve fracture to assist in sleeve retraction. The sleeve may be used singly or in pairs with either self-expanding or balloon expandable stents. In the case of a self expanding stent, one or more sleeves may be utilized in conjunction with one or more retractable sheaths. The sleeve(s) may be provided in a variety of lengths to provide partial to full stent coverage. Other inventive aspects and embodiments of the present end retaining sleeves will be made apparent below.

### BRIEF DESCRIPTION OF THE SEVERAL VIEWS OF THE DRAWINGS

A detailed description of the invention is hereafter described with specific reference being made to the drawings in which:

FIG. 1 is a side perspective view of a first embodiment of the invention;

FIG. 2 is a side perspective view of a second embodiment of the invention;

FIG. 3 is a side perspective view of a third embodiment of the invention;

FIG. 4 is a side perspective view of a fourth embodiment of the invention;

FIG. 5 is a side perspective view of a fifth embodiment of the invention;

FIG. 6 is a side perspective view of a sixth embodiment of the invention;

FIG. 7 is a side perspective view of the embodiment shown in FIG. 6 as may be seen during stent expansion;

FIG. 8 is a side perspective view of the embodiment shown in FIG. 7 as may be seen during sleeve retraction; and

FIG. 9 is a side perspective view of the embodiment of the invention.

### DETAILED DESCRIPTION OF THE INVENTION

While this invention may be embodied in many different forms, there are shown in the drawings and described in detail herein specific preferred embodiments of the invention. The present disclosure is an exemplification of the principles of the invention and is not intended to limit the invention to the particular embodiments illustrated.

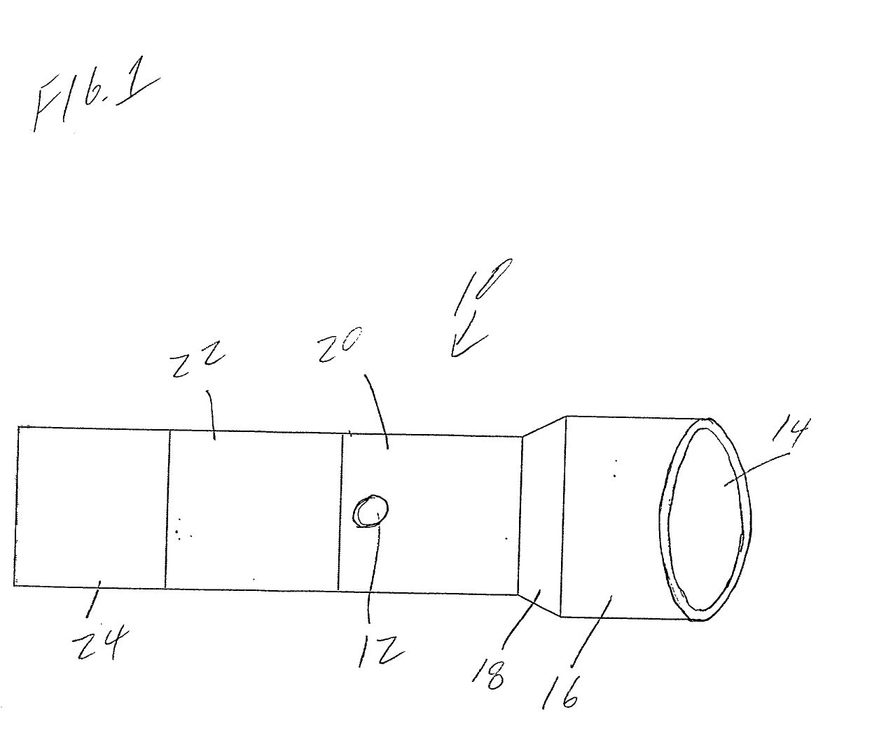

As may be seen in FIG. 1, the present invention may be embodied in an elastomeric sock or sleeve 10 having one or more ports 12 which may be utilized to inject or otherwise place a lubricious substance (not shown) on to the inside surface 14 of the sleeve 10. Sleeves such as sleeve 10 presently described, may be characterized as having a stent overlaying portion 16, a cone portion 18, a cone waist transition portion 20, a waist portion 22 and a balloon end portion 24.

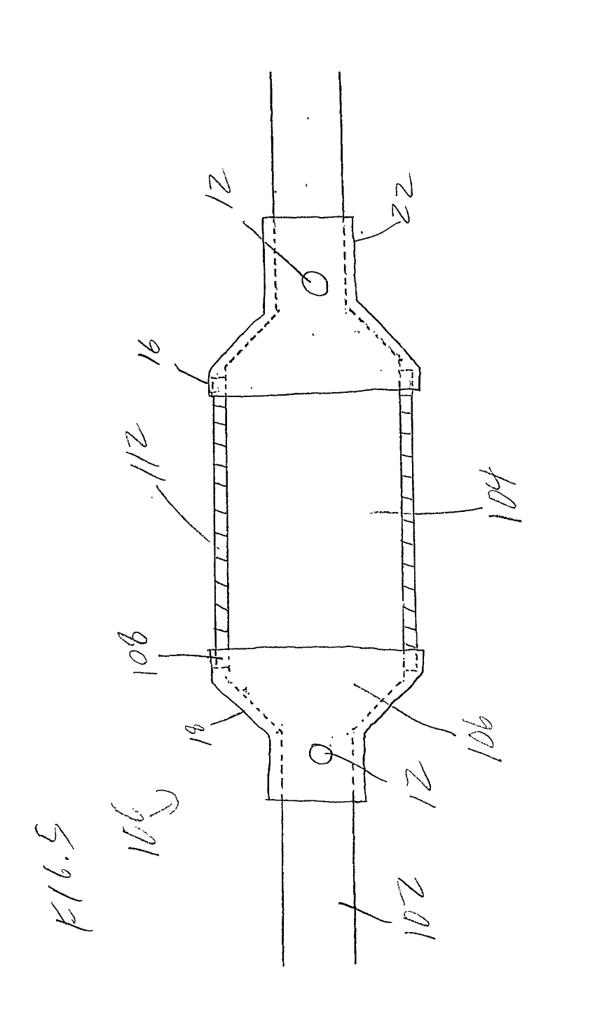

The port(s) 12 may be located anywhere on the sleeve 10. However, as may be seen in the embodiment shown in FIG. 1, the port(s) 12 are positioned immediately adjacent to the waist portion 22, and through a portion of the cone waist transition portion 20. Because the waist portion 22 may be engaged to a catheter shaft, the position of the port(s) 12 in the embodiment shown in FIG. 1, ensures that a lubricant inserted therethrough will have maximum coverage of the inside surface 14 of the sleeve 10 where it overlays a respective balloon cone 106 and stent end 108, such as may best be seen in FIG. 5.

When the sleeve 10 is used in conjunction with a stent delivery catheter 100 such as may be seen in FIG. 5, the port(s) 12 are preferably located immediately adjacent to the section of the sleeve 10, such as the waist portion 24 where the sleeve 10 is engaged or bonded to the catheter shaft 102. Where the balloon 104 is integral with the shaft 104 the port 12 may be adjacent to the balloon end portion 24.

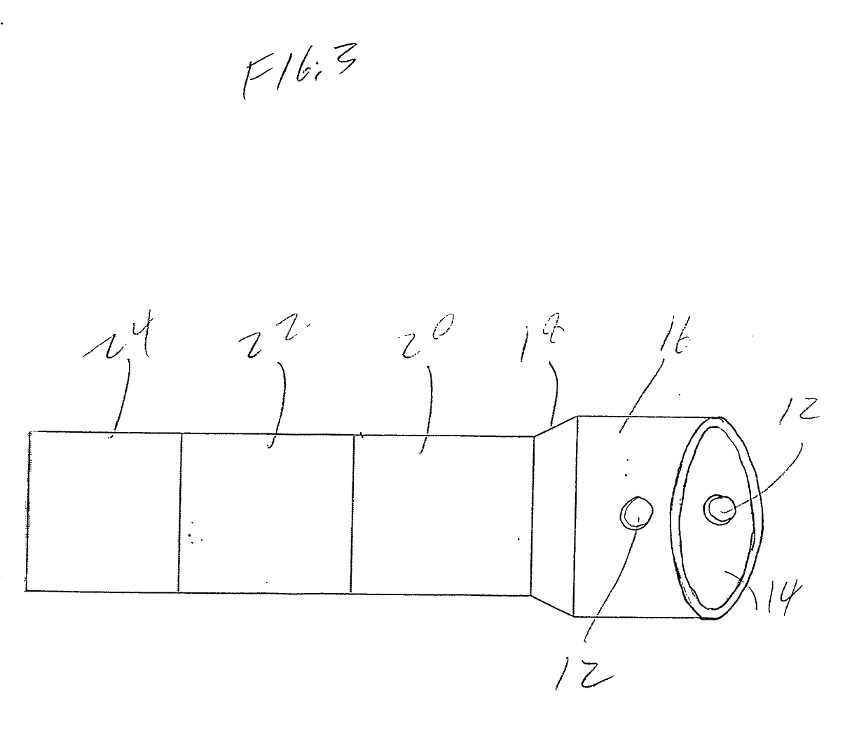

While the relative positions of ports 12 shown in FIG. 1 and FIG. 5 may be preferred. The ports 12 may be placed in an infinite variety of positions and combinations. It should also be noted that while a single port 12 may be used, two or more opposingly positioned ports 12 may be utilized such as are illustrated in FIG. 3. In addition a variety of ports, which vary in number, shape and position may also be utilized.

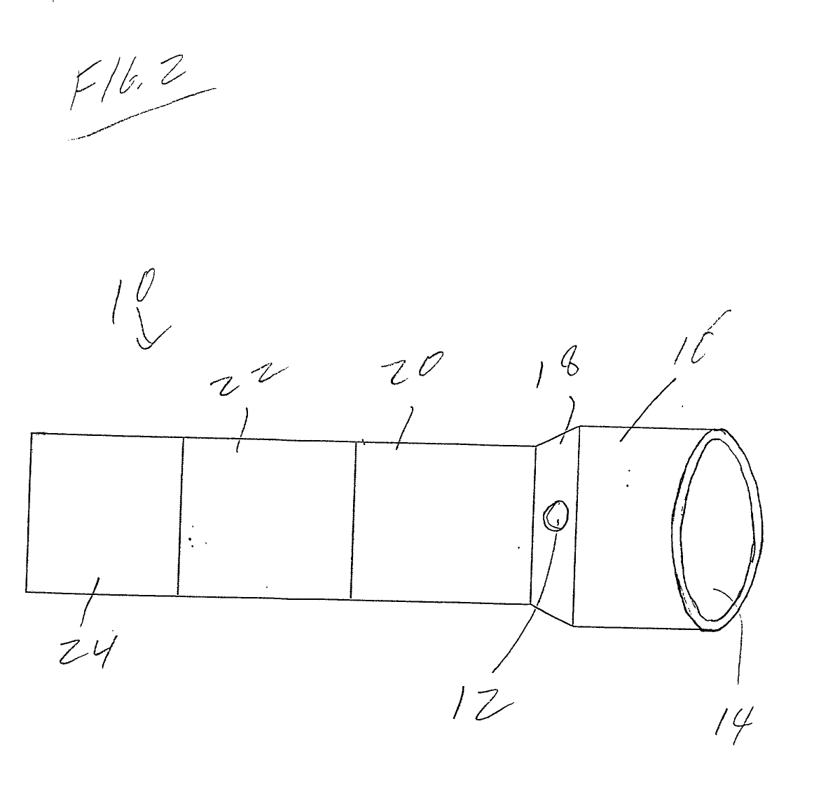

Some examples of alternative port placement are provided as follows: As may be seen in FIG. 2, the port(s) 12 is defined by the cone portion 18. Such port 12 placement may be desired to help ensure lubricant application to the balloon cone 106 (such as may be seen in FIG. 5) which may lie thereunder.

FIG. 3 shows an embodiment of the sleeve 10 wherein the ports 12 are defined by the stent overlaying portion 16. Such port 12 placement may be desired to help ensure lubricant application to the stent end 108 (such as may be seen in FIG. 5) which may lie thereunder.

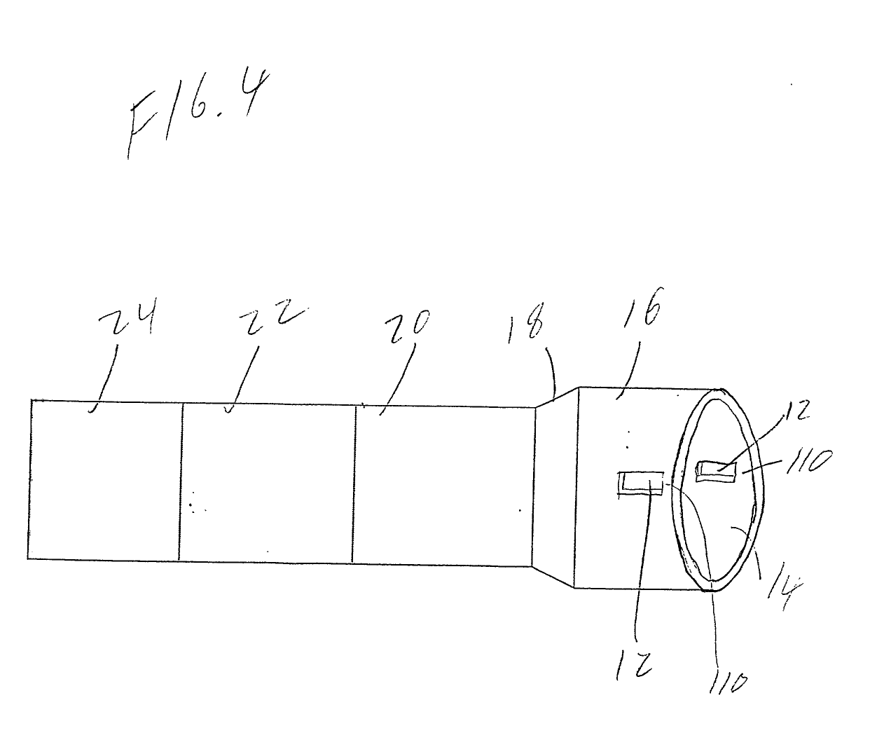

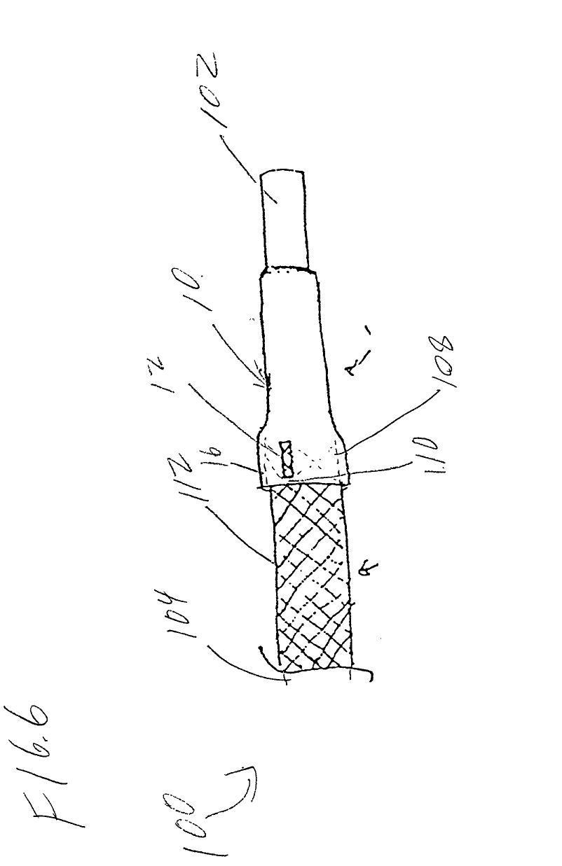

As previously indicated, port(s) 12 may have a variety of shapes and sizes as well as positions. As may be seen in FIG. 4, a pair of ports 12 are shown having a rectangular configuration, however any shapes may also be used. In the embodiment shown the ports 12 are defined by the stent overlaying portion 16. The ports 12 may be configured to fracture or otherwise tear the thin portion of sleeve material 110 which defines at least one side of the port. When a sleeve 10 is used in conjunction with a stent delivery catheter 100 such as shown in FIG. 6, the ports 12 may be positioned in whole or in part over the stent ends 108.

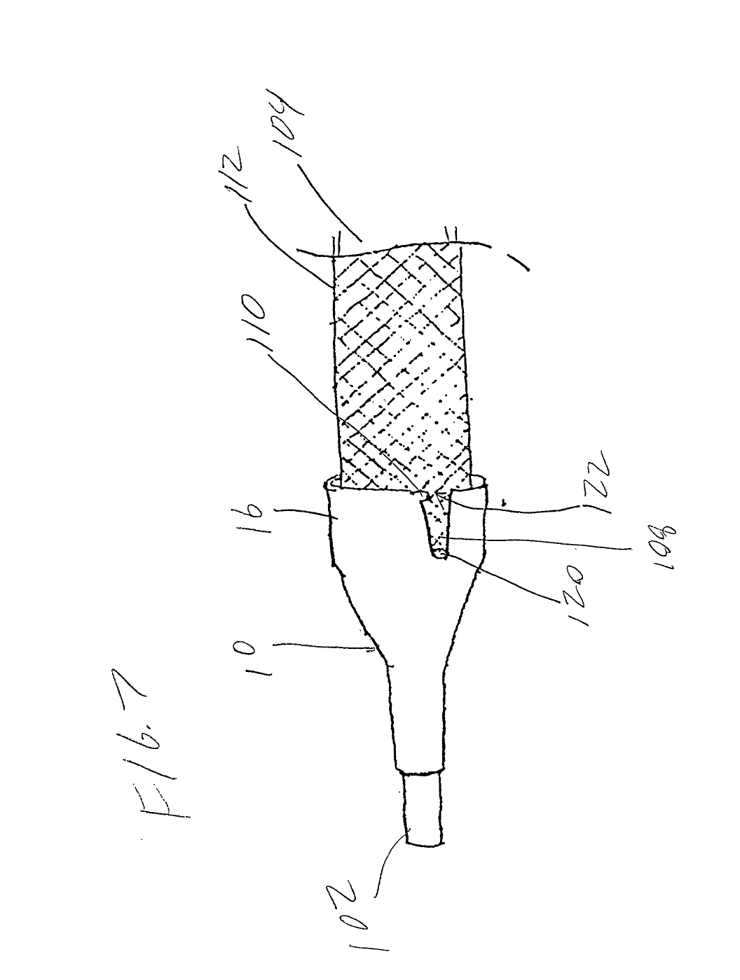

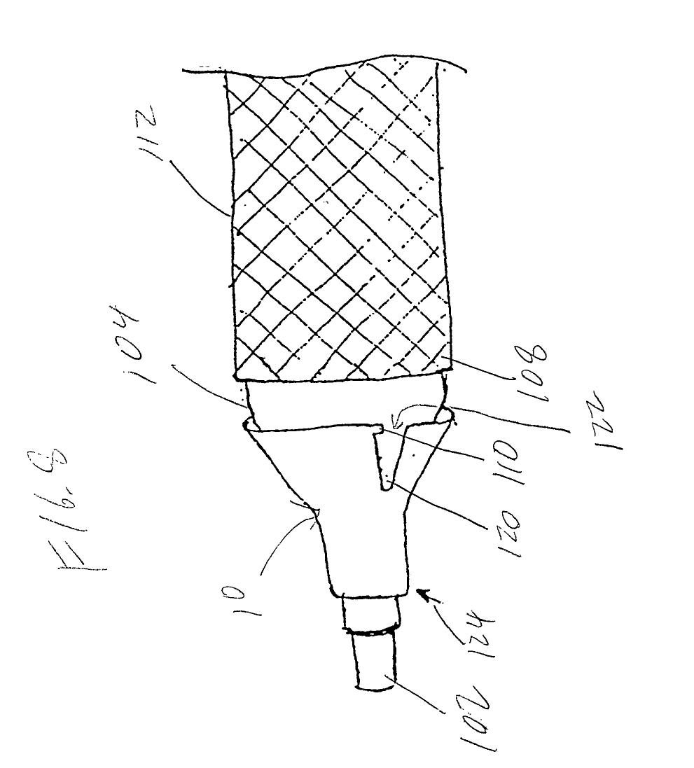

The balloon 104 and stent 112 may be expanded from an unexpanded state, such as may be seen in FIG. 6, to an expanded state, such as may be seen in FIG. 8. During stent 112 expansion, one or more portions of the sleeve 10, most notably the stent overlaying portion 16 may also have a predetermined amount of outward pressure exerted on it. The thin portion of material 110 is constructed and arranged to rupture when a predetermined amount of outwardly acting force is exerted against at least a portion of the sleeve 10 such as is exerted against the sleeve 10 during stent 112 expansion, such as may be seen in FIG. 7.

As is shown in FIG. 7, during expansion the rupture of the thin portion of material 110 result in a slot 120 having an opening 122. The opening 122 of the slot 120 may be characterized as a break in the sleeve 10 which allows at least the stent overlaying portion 16 to split thereby providing for a sudden increase in stent overlaying portion 16 diameter and a resulting reduction in frictional engagement and radial compression which would otherwise be provided by the stent overlaying portion 16 of the sleeve 10 against the expanding stent 112. This reduces radial sleeve strength thereby allowing the sleeve 10 to more readily retract off of the stent ends 108, such as may best be seen in FIG. 8, thereby freeing the stent 112 from under the sleeve 10 as a result of expansion.

As may also be seen in FIG. 8, after the material 110 breaks or ruptures in the manner described above, the sleeve 10 is constructed and arranged to retract off of the stent ends 108 in the manner shown and indicated by reference arrow 124.

The sleeve 10 may be retracted off of the stent 112 in the folding configuration indicated by arrow 124 or the stent may roll up on itself along the catheter shaft 102. It should be noted however, that any type of retraction mode may be attributed to the sleeves 10, in addition or instead of the configuration shown and described.

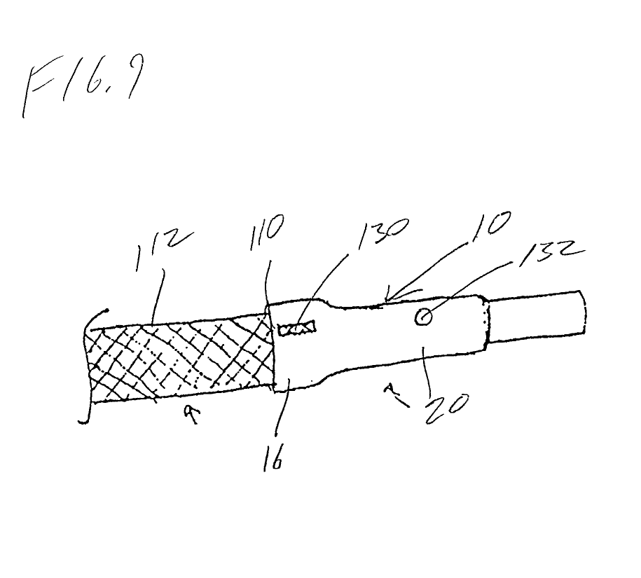

Turning to FIG. 9, an alternative embodiment of the invention is illustrated wherein two different types of lubricant injection ports are positioned on a stent retaining sleeve 10. As shown a first tear-away port 130 is defined by the thin material 110 and the stent overlaying portion 16. A second port 132 is defined by the cone waist transition portion 20. By providing the sleeve 10 with multiple lubricant filler ports the inside surface 14 of the sleeve 10 may be more throughly lubricated. In addition, the tear-away port 132 helps to provide controlled sleeve rupture for controlled stent 112 release such as previously described.

In addition to being directed to the embodiments described above and claimed below, the present invention is further directed to embodiments having different combinations of the features described above and claimed below. As such, the invention is also directed to other embodiments having any other possible combination of the dependent features claimed below.

The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

1. A stent delivery system comprising:
a stent delivery catheter, the stent delivery catheter having a balloon mounted thereon, the balloon having an unexpanded position and an expanded position, the balloon having at least one waist portion, at least one cone portion and at least one body portion;
a stent disposed about the at least one body portion, the stent having an expanded state and an unexpanded state;
at least one sleeve, the at least one sleeve having a waist overlay portion, a cone overlay portion, and a stent overlay portion, when the stent is in the unexpanded state the waist overlay portion being engaged to at least a portion of the at least one waist portion and the cone overlay portion being disposed about the at least one cone portion and the stent overlay portion being disposed about at least a portion of the stent,
the at least one sleeve having at least one lubricant application port, the at least one lubricant application port defining an opening through the at least one sleeve, the opening constructed and arranged to allow a lubricious substance to pass through the at least one sleeve and at least partially coat a portion of the balloon.

2. The stent delivery system of claim 1 wherein the at least one lubricant application port is at least partially defined by the waist overlay portion.

3. The stent delivery system of claim 1 wherein the at least one lubricant application port is at least partially defined by the cone overlay portion.

4. The stent delivery system of claim 3 wherein the at least one lubricant application port is at least partially defined by a portion of the stent overlay portion.

5. The stent delivery catheter of claim 4 wherein the portion of the stent overlay portion is constructed and arranged to fracture during expansion of the stent from the unexpended state to the expanded state.

6. The stent delivery system of claim 5 wherein the at least one lubricant application port further comprises at least one tear-away lubricant application port and at least one standard lubricant application port, the at least one tear-away lubricant application port at least partially defined by the portion of the stent overlay portion, the at least one lubricant application port being positioned elsewhere through the at least one sleeve.

7. The stent delivery system of claim 5 wherein the at least one sleeve is constructed and arranged to retract off of a stent subsequent to the fracture of the portion of the stent overlay portion.

8. An elastomeric sleeve constructed and arranged to retain a stent in the unexpanded state on a stent delivery catheter, the elastomeric sleeve comprising:
an inside surface, and at least one lubricant application port
the at least one lubricant application port defining an opening through the at least one sleeve, the opening constructed and arranged to allow a lubricious substance to pass through the at least one sleeve an coat at least a portion of the inside surface.

9. The elastomeric sleeve of claim 8 further comprising a first sleeve and a second sleeve, the first sleeve and second sleeve each constructed and arranged to be disposed about an end of a stent delivery balloon.
